(19) United States
(12) Patent Application Publication
(10) Pub. No.: US 2024/0102090 A1
(43) Pub. Date: Mar. 28, 2024

(54) METHOD FOR MULTIMODAL PROFILING OF INDIVIDUAL EXTRACELLULAR VESICLES

(71) Applicant: WellSIM Biomedical Technologies, Inc., San Jose, CA (US)

(72) Inventors: Yuchao CHEN, San Jose, CA (US); Ching-Fang CHANG, San Jose, CA (US)

(21) Appl. No.: 18/459,391

(22) Filed: Aug. 31, 2023

Related U.S. Application Data
(60) Provisional application No. 63/409,743, filed on Sep. 24, 2022.
(60) Provisional application No. 63/415,656, filed on Oct. 13, 2022.

(51) Int. Cl. C12Q 1/6869; C12Q 1/6804; G01N 33/569

(52) U.S. Cl. CPC C12Q 1/6869; C12Q 1/6804; G01N 33/56966; C12Q 2600/158; C12Q 2600/16; G01N 2333/705

(57) ABSTRACT

The present disclosure provides a methodology for multimodal profiling of extracellular vesicles at single-particle resolution, including technique, workflow, and analytical algorithm.

### CROSS-REFERENCE TO RELATED APPLICATIONS

This application claims the priority of U.S. provisional application 63/409,743, filed Sep. 24, 2022, and U.S. provisional application 63/415,656, filed Oct. 13, 2022, the entire disclosure of which is incorporated herein by reference.

### FIELD OF THE INVENTION

The present invention generally relates to methods for detecting extracellular vesicles. More particularly, the invention relates to systems and methods for multimodal profiling of individual extracellular vesicles.

### BACKGROUND OF THE INVENTION

Extracellular vesicles (EVs) are lipid bilayer nanoparticles secreted by most cell types, which encapsulate a subset of biomolecules from their cells of origin, including lipids, proteins, and nucleic acids. EVs play a vital role in intercellular communication with many physiological and pathological implications. Therefore, profiling of EV-carried biomolecules (e.g., RNA, DNA, protein, and lipid) provides a promising approach for early diagnosis of various diseases, such as cancers, neurodegenerative diseases, and autoimmune diseases.

However, traditional EV characterization methods, such as western blot, ELISA, mass spectrometry (MS), and next-generation sequencing (NGS), usually analyze entire EV population (bulk-level analysis) and have several unavoidable limitations. First, EVs are highly heterogeneous in their molecular composition, especially when secreted by different cell types. Bulk-level analysis is not able to reveal the heterogeneity of EVs. It is also difficult to use bulk-level analysis to identify and quantify each EV subtype in a complex biofluid. Second, in plasma, extracellular vesicles constitute up to only 10% of all nanoparticles, and only a small proportion of all extracellular vesicles are disease specific. The sensitivity of bulk-level analysis method is unsatisfactory for identifying the small number of disease-specific EVs. Moreover, in bulk-level analysis, impurities (e.g., nucleic acids and proteins) and concentration variation between EV samples limit the accuracy of the analysis results. Therefore, EV analysis at the single particle level has become of increasing interest to the researchers.

Currently, some techniques have been developed for profiling surface proteins of individual EVs, mainly including imaging-based and sequencing-based approaches. The imaging-based method, detecting the fluorescent signals of individual EVs via a high-resolution camera or photosensor, usually can only detect limited types of surface proteins (typically <5). Wu et al. reported a sequencing-based proximity barcoding assay, which can overcome this limitation (Wu, D. et al. Profiling surface proteins on individual EVs using a proximity barcoding assay. Nat. Commun., 10, 3854 (2019)). However, the EVs were barcoded in the same aqueous environment, raising a concern of mislabeling. Furthermore, genomic and transcriptomic profiling of individual EVs have not yet been reported so far, leaving an unmet gap in EV study.

Therefore, traditional bulk-level analysis methods fail to represent their individual variations. A methodology for multimodal profiling of EVs at single-particle resolution, including technique, workflow, and analytical algorithm, is of great interest to the EV research field, but has yet to be developed.

### BRIEF SUMMARY OF THE INVENTION

In one aspect, this disclosure provides a method of multimodal profiling of an extracellular vesicle (EV) at single-EV resolution. In one embodiment. the method comprises:
a) obtaining a biological sample comprising the EV, wherein the EV contains a surface antigen and a target nucleic acid;
b) incubating the EV with an antibody-nucleic acid conjugate comprising (1) an antibody that binds to the surface antigen, and (2) a nucleic-acid based antibody tag, thereby linking the antibody-nucleic acid conjugate to the EV;
c) purifying the EV to remove excess antibody-nucleic acid conjugate not linking to the EV;
d) mixing the labeled EV and a barcoded bead for conjugation, wherein the barcoded bead comprise a bead conjugated with a nucleic-acid based bead tag;
e) encapsulating the EV-bead conjugate and EV lysis buffer into a droplet or a microwell
f) lysing the EV to release the target nucleic acid and the antibody tag; and
g) sequencing the target nucleic acid, the antibody tag, and the bead tag to profile the EV at single-EV resolution.

In another embodiment, the method comprises:
a) obtaining a biological sample comprising the EV, wherein the EV contains a surface antigen and a target nucleic acid.
b) incubating the EV with an antibody-nucleic acid conjugate comprising (1) an antibody that binds to the surface antigen, and (2) a nucleic-acid based antibody tag, thereby linking the antibody-nucleic acid conjugate to the EV;
c) purifying the EV to remove excess antibody-nucleic acid conjugate not linking to the EV;
d) encapsulating the labeled EV, a barcoded bead, and EV lysis buffer into a droplet, wherein the barcoded bead comprises a bead conjugated with a nucleic-acid based bead tag;
e) lysing the EV encapsulated in step (d) to release the target nucleic acid and the antibody tag to the droplet; and
f) sequencing the target nucleic acid, the antibody tag, and the bead tag to profile the EV at single-EV resolution.

In some embodiments, the target nucleic acid is mRNA, microRNA, lncRNA, tRNA, snRNA, YRNA or vault RNA. In some embodiments, the target nucleic acid is mRNA.

In some embodiments, the surface antigen is selected from the group consisting of CD56, CD171, CD9, CD63, CD81, GPC1, FN, PSMA, CD30, FoxP3, CCR8, SiglecF, Ly6G, CCL3, ga13, t-Tau, p-Tau, A1340, and A1342.

In some embodiments, the antibody tag comprises an antibody barcode region, and an antibody hybridization region. In some embodiments, the antibody hybridization region is a poly dA tail.

In some embodiments, the purifying step involves an ultrafiltration-based method. In some embodiments, the ultrafiltration-based method is tangential flow filtration. In some embodiments, the ultrafiltration-based method is EXODUS (EV detection via the ultrafast-isolation system).

In some embodiments, the encapsulating step involves a microfluidics method.

In some embodiments, the ratio of barcoded bead to labeled EV is about 1.5.

In some embodiments, labeled EV is at a concentration of about 1000 EV/μL and the barcoded bead is at a concentration of 1500 beads/μL in EV lysis buffer.

In some embodiments, a flow rate of 15 mL/h was used for oil phase, and 4 mL/h was used for both barcoded bead and labeled EV.

In some embodiments, wherein the nucleic-acid based bead tag comprises a PCR handle, an EV barcode region, a unique molecular identifier (UMI) region and a target binding region.

In some embodiments, the target binding region is a poly dT tail. In some embodiments, the target binding region is a random sequence. In some embodiments, the target binding region is a random hexamer. In some embodiments, the target binding region is combination of poly dT tail and random hexamer.

In another embodiment, the method comprises:
a) obtaining a biological sample comprising the EV, wherein the EV contains a surface antigen and a target nucleic acid.
b) incubating the EV with an antibody-nucleic acid conjugate comprising (1) an antibody that binds to the surface antigen, and (2) a nucleic-acid based antibody tag, thereby linking the antibody-nucleic acid conjugate to the EV;
c) purifying the EV to remove excess antibody-nucleic acid conjugate not linking to the EV;
d) incubating the EV with a barcoded bead, wherein the barcoded bead comprises a bead conjugated with a nucleic-acid based bead tag, thereby forming an EV-antibody-nucleic acid conjugate-barcoded bead composition;
e) partitioning the composition in step (d);
f) lysing the partitioned composition in step (e) to release the target nucleic acid and the antibody tag; and
g) sequencing the target nucleic acid, the antibody tag, and the bead tag to profile the EV at single-EV resolution.

In some embodiments, the partitioning step comprises separating the bead by droplet generation. In some embodiments, the droplet generation is accomplished by microfluidics method. In some embodiments, the partitioning step comprises separating the composition into microwells. In some embodiments, separating the composition into the microwells comprises introducing the composition into a support comprising the microwells. In some embodiments, the microwells have a size of about 500 nL or less.

In some embodiments, the barcoded bead is a magnetic bead, optionally wherein the barcoded bead comprises ferrite or magnetite (Fe₃O₄)

In some embodiments, the ratio of barcoded bead to EV is larger than 1. In some embodiments, the ratio of barcoded bead to EV is larger than 2. In some embodiments, the ratio of barcoded bead to EV is larger than 5. In some embodiments, the ratio of barcoded bead to EV is larger than 10.

In another aspect, the invention further provides devices and systems that find use in practicing embodiments of the invention. In some embodiments, the devices or include a) EXODUS purification module, b) encapsulation module, c) sequencing module and d) data analysis module.

In another aspect, the invention further provides a diagnostic method using the method of multimodal profiling of an extracellular vesicle (EV) at single-EV resolution described herein.

These and other features, aspects, and advantages of the present invention will become better understood with regard to the following description, appended claims and accompanying drawings.

### BRIEF DESCRIPTION OF THE FIGURES

The following drawings form part of the present specification and are included to further demonstrate certain aspects of the present disclosure. The disclosure may be better understood by reference to one or more of these drawings in combination with the detailed description of specific embodiments presented herein.

FIG. 1 shows the incubation of EV with an antibody-nucleic acid conjugate, wherein the antibody-nucleic acid conjugate comprising (1) an antibody that binds to the surface antigen, and (2) a nucleic-acid based antibody tag.

FIG. 2 shows the purification of the EV using the EXODUS.

FIG. 3 shows a typical procedure of microfluidic droplet generation.

FIG. 4 shows the barcoded bead which comprises a bead tag conjugated to a bead, wherein the bead tag comprising barcoded bead comprise a bead conjugated with a nucleic-acid based bead tag.

FIG. 5 shows the target nucleic acid of the EV released under lysis buffer.

FIG. 6 shows the association between the bead tag's target binding region and the target nucleic acid or antibody tag.

FIG. 7 shows the partitioning of the EV-antibody-bead composition.

FIG. 8 shows the proposed EXODUS-EXOSeq-NGS workflow for multimodal profiling of individual EVs.

FIG. 9 shows the workflow for integrative proteomic and transcriptomic profiling of individual EVs.

FIG. 10A shows the results of NTA measurement of the concentration and size distribution of purified human and mouse EVs. FIG. 10B shows the generation of emulsion droplets via a flow-focusing droplet generator. FIG. 10C shows the analysis of a mixture of mouse and human EVs. FIG. 10D shows tSNE clustering of human and mouse EVs. FIG. 10E shows the top 5 expressed genes.

FIG. 11 shows the workflow of single-EV capture via barcode beads.

FIG. 12 shows the workflow of library preparation for single-EV sequencing for profiling of single-EV gene expression and surface markers for integrative proteomic and transcriptomic profiling of individual EVs.

FIG. 13A shows the results of TapeStation analysis of the library comprising antibody tag and target nucleic acid. FIG. 13B and FIG. 13C show the analysis of mixture of mouse and human EVs in terms of surface protein and gene expression. FIG. 13D shows the analysis of human EVs in terms of CD81 and CD63 surface antigens. FIG. 13E shows the expression of CD9 protein and TAOK1 gene in a UMAP clustering.

### DETAILED DESCRIPTION OF THE INVENTION

EVs, small extracellular vesicles (30-200 nm), are highly heterogeneous in biofluids. However, traditional bulk-level analysis methods fail to represent their individual variations. A methodology for multimodal profiling of EVs at single-particle resolution, including technique, workflow, and analytical algorithm, is of great interest to the EV research field, but has yet to be developed.

The disclosure provides methods for multimodal profiling of an extracellular vesicle (EV) at single-EV resolution. More particularly, this disclosure provides methods a multimodal profiling method for perform transcriptomic and proteomic analysis at single-EV level with improved operation simplicity and accuracy.

This technique will address these unmet needs, which could significantly accelerate the development of new techniques for EV-based disease diagnosis and targeted therapies. This technique, which can provide unprecedented resolution and sensitivity for EV interrogation and offer important biological information that is missed in bulk-level analysis, enables the reduction of biological noise and offers the ability to investigate and characterize rare EV subtypes. This method will not only develop a technique for multimodal analysis of individual EVs, but also will establish a bioinformatics data model of different EV subtypes, enabling rapid identification of their subpopulations and origins. Transcriptome and genome profiling at single-EV resolution will reveal the variations of their nucleic acids contents and improve the understanding on their biogenesis and specific biological functions.

In one aspect, this disclosure provides method of multimodal profiling of an extracellular vesicle (EV) at single-EV resolution. In one embodiments, the method comprises:
a) obtaining a biological sample comprising the EV, wherein the EV contains a surface antigen and a target nucleic acid;
b) incubating the EV with an antibody-nucleic acid conjugate comprising (1) an antibody that binds to the surface antigen, and (2) a nucleic-acid based antibody tag, thereby linking the antibody-nucleic acid conjugate to the EV;
c) purifying the EV to remove excess antibody-nucleic acid conjugate not linking to the EV;
d) mixing the labeled EV and a barcoded bead for conjugation, wherein the barcoded bead comprise a bead conjugated with a nucleic-acid based bead tag;
e) encapsulating the EV-bead conjugate and EV lysis buffer into a droplet or a microwell
f) sequencing the target nucleic acid, the antibody tag, and the bead tag to profile the EV at single-EV resolution.

In another embodiment, the method multimodal profiling of EV comprises:
a) obtaining a biological sample comprising the EV, wherein the EV contains a surface antigen and a target nucleic acid;
b) incubating the EV with an antibody-nucleic acid conjugate comprising (1) an antibody that binds to the surface antigen, and (2) a nucleic-acid based antibody tag, thereby linking the antibody-nucleic acid conjugate to the EV;
c) purifying the EV to remove excess antibody-nucleic acid conjugate not linking to the EV;
d) encapsulating the labeled EV, a barcoded bead, and EV lysis buffer into a droplet, wherein the barcoded bead comprise a bead conjugated with a nucleic-acid based bead tag;
e) lysing the EV encapsulated in step (d) to release the target nucleic acid and the antibody tag to the droplet; and
f) sequencing the target nucleic acid, the antibody tag, and the bead tag to profile the EV at single-EV resolution.

Obtaining a Biological Sample Comprising the EV

As used herein, the term “biological sample” or “sample” refers to a sample obtained from a laboratory, such as cultured cells. “Biological sample” or “sample” can also refer to a fluid or tissue obtained from a patient. For instance, any bodily fluid, e.g., blood, urine, saliva, cerebrospinal fluid (CSF), cyst fluids, or fluid obtained from a lavage. Alternatively, a biological sample can be obtained from tissues or organs.

In some embodiments, the biological sample is selected from the group consisting of blood, plasma, serum, urine, sputum, spinal fluid, cerebrospinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid and combinations thereof. In some embodiments, the biological sample is plasma or serum.

Plasma and serum are rich in EVs derived from different sources. Even if EVs could be isolated from complex biofluids (e.g., whole blood), only a small portion of them are disease-specific. The present invention can reduce the complexity of EVs, and detect EVs derived from certain cells at low abundance In some embodiments, the EV is derived from from neuronal cells. In some embodiments, the biological sample is from a patient with a neurodegenerative disease, e.g., Parkinson's disease (PD) or Alzheimer's disease (AD).

The invention can provide unprecedented resolution and sensitivity for EV interrogation and offer important biological information that is missed in bulk-level analysis, enables the reduction of biological noise and offers the ability to investigate and characterize rare EV subtypes.

In some embodiments, the “target nucleic acid” described herein comprises DNA or RNA. Examples of RNA include messenger RNAs, long non-coding RNAs, transfer RNAs, ribosomal RNAs, small RNAs (non-protein-coding RNAs, non-messenger RNAs), microRNAs, piRNAs, snRNAs, snoRNAs, and Y-RNAs.

As used herein, the term “nucleic acids” refer to DNA and RNA unless otherwise specified. The nucleic acids can be single stranded or double stranded. In some instances, the nucleic acid is DNA. In some instances, the nucleic acid is RNA. RNA includes, but is not limited to, messenger RNA, transfer RNA, ribosomal RNA, non-coding RNAs, microRNAs, and HERV elements.

Incubating the EV with an Antibody-Nucleic Acid Conjugate

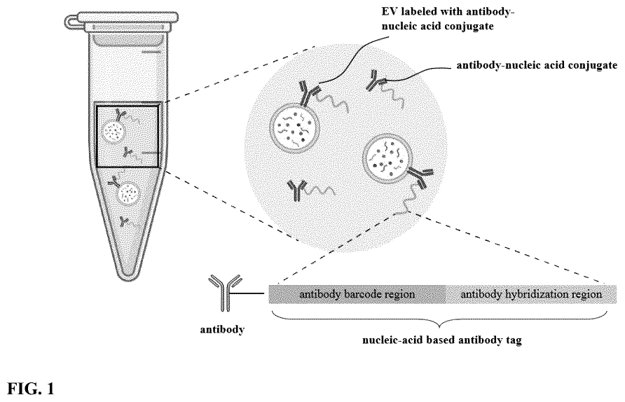

As shown in FIG. 1, the EV is incubated with an antibody-nucleic acid conjugate, wherein the antibody-nucleic acid conjugate comprising (1) an antibody that binds to the surface antigen, and (2) a nucleic-acid based antibody tag.

Any antibody that is capable of specifically binding to the surface antigen of the EV can be used. In some embodiments, the surface antigen is selected from the group consisting of CD56, CD171, CD9, CD63, CD81, GPC1, FN, PSMA, CD30, FoxP3, CCR8, SiglecF, Ly6G, CCL3, ga13, t-Tau, p-Tau, A1340, and A1342.

The antibodies as used herein can be either full-length immunoglobulin molecules or immunologically active moieties, such as antibody fragments. In some embodiments, the antibody is a functional antibody fragment. For example, an antibody fragment can be part of an antibody, such as F(ab′)2, Fab′, Fab, Fv, sFv, and the like.

The antibody tag comprises an antibody barcode region, and an antibody hybridization region. The antibody hybridization region is capable of hybridize with the target binding region in the bead tag of the barcoded beads. In some embodiments, the antibody hybridization region is a non-specific sequence such as poly(A) tail. In some embodiments, the antibody hybridization region is a specific sequence.

The antibody barcode region can comprise a nucleic acid sequence that provides information of the antibody binding to the EV.

Purifying

Once the EVs have been labelled with the antibody-nucleic acid conjugates, labelled EVs are purified by removing unbound antibody-nucleic acid conjugates. In some embodiments, the EV is purified by method such as ultracentrifugation, ultrafiltration, size exclusion chromatography.

In some embodiments, the purifying step involves an ultrafiltration-based method. In some embodiments, the ultrafiltration-based method is tangential flow filtration. In tangential flow filtration, a feed stream is made to flow parallel to a porous membrane to avoid clogging, as occurs in dead-end filtration.

In some embodiments, the ultrafiltration-based method is based on EXODUS (EV detection via the ultrafast-isolation system). EXODUS is a platform for ultrafast, high-performance EV purification. The applicant surprisingly found that, by introducing double coupled harmonic oscillations into a dual-membrane filter configuration for the generation of transverse waves, the oscillation of nanoporous membranes and cartridge substantially inhibits fouling effects, which can otherwise lead to a decrease in the permeated flux, via acoustofluidic streaming, resulting in improved processing speed, yield and purity. Based on this mechanism, the applicant incorporates the EXODUS into the purification of the labeled EV, which allows a simplified operation and superior robustness and reproducibility.

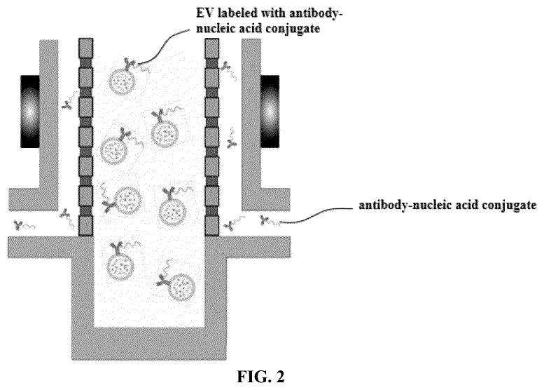

As illustrated in FIG. 2, the EXODUS purifies the EV to remove excess antibody-nucleic acid conjugate not linking to the EV. Details of EXODUS is described by Chen Y C et al. (EV detection via the ultrafast-isolation system: EXODUS, Nature Methods, 18, 2021, 212-218).

The purified EV is then subjected to droplet generation. In some embodiments, droplet generation is passive droplet generation, which can be performed using certain microfluidic geometries, namely, cross-flowing, flow focusing, and co-flowing.

In some embodiments, the microfluidic method for droplet generation is flow focusing droplet generation. Flow focusing involves the dispersed phase flowing to meet the continuous phase typically at an angle (nonparallel streams). The dispersed phase then undergoes a constraint that creates a droplet. The constraint is typically a narrow channel, which creates the droplet though symmetric shearing. Slower the flow rate, bigger is the droplet size, and vice versa. Additional details about flow focusing droplet generation are well known to a person of ordinary skill in the art and such embodiments are within the purview of the invention.

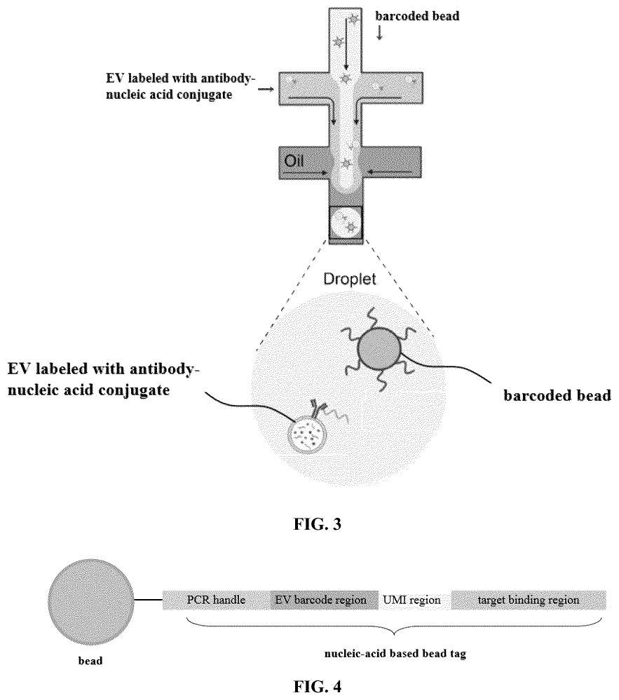

A typically procedure of microfluidic droplet generation is shown in FIG. 3. The labeled EV and barcoded bead are mixed together with a lysis buffer and be used as the droplet phase. Meanwhile, a continuous oil phase is provided. The droplet phase flowing to meet the continuous oil phase typically at an angle (perpendicular). The droplet phase then undergoes a constraint that creates a droplet.

Details about the droplet generation step depend on the intended size of the droplet, the type of sample tested, the content of biomarkers in the EVs, etc. Particularly, the parameters for performing the microfluidic droplet generation is critical for EV profiling. For the methods disclosed herein to function as intended, less than about 5%, preferably, less than about 4%, more preferably, less than about 3%, even more preferably, less than about 2%, and most preferably, less than about 1% of the compartments contain two or more beads. Ideally, none of the compartments contains two or more beads.

In some embodiments, the ratio of barcoded bead to labeled EV is about 1.5. In some embodiments, the ratio of barcoded bead to labeled EV is about 1.4. In some embodiments, the ratio of barcoded bead to labeled EV is about 1.3. In some embodiments, the ratio of barcoded bead to labeled EV is about 1.2. In some embodiments, the ratio of barcoded bead to labeled EV is about 1.1.

In some embodiments, the labeled EV is at a concentration of about 1000 EV/μL and the barcoded bead is at a concentration of 1500 beads/μL in EV lysis buffer.

In some embodiments, a flow rate of 15 mL/h was used for oil phase, and 4 mL/h was used for both barcoded bead and labeled EV.

Barcoded Bead

As shown in FIG. 4, the barcoded bead comprises a bead conjugated with a nucleic-acid based bead tag.

In some embodiments, the nucleic-acid based bead tag comprises a PCR handle, an EV barcode region, a unique molecular identifier (UMI) region and a target binding region.

In some embodiments, the PCR handle comprises a nucleic acid sequence that is capable of hybridizing to a sequencing primer. In some embodiments, a PCR handle can comprise a nucleic acid sequence that is capable of hybridizing to a PCR primer. In some embodiments, the PCR handle can comprise a nucleic acid sequence that is capable of hybridizing to a sequencing primer and a PCR primer.

The PCR handle can comprise a sequence that can be used to initiate transcription of the bead tag. A PCR handle can comprise a sequence that can be used for extension of the bead tag or a region within the bead tag. A PCR handle can be, or be about, 1, 2, 3, 4, 5, 10, 15, 20, 25, 30, 35, 40, 45, 50, or a number or a range between any two of these values, nucleotides in length. For example, a PCR handle can comprise at least about 10 nucleotides. A PCR handle can be at least, or be at most, 1, 2, 3, 4, 5, 10, 15, 20, 25, 30, 35, 40, 45, 50, 100, 200, or 300 nucleotides in length. In some embodiments, a cleavable linker or modified nucleotide can be part of the PCR handle sequence to enable the bead tag to be cleaved off from the support.

In some embodiments, the EV barcode region can comprise a nucleic acid sequence that provides information for determining which target nucleic acid originated from which EV, or which antibody is bind to which EV's surface antigen. In some embodiments, the EV barcode region is identical for all bead tag attached to a given solid support (e.g., a bead), but different for different solid supports (e.g., beads).

In some embodiments, the UMI region can comprise a nucleic acid sequence that provides identifying information for the specific type of target nucleic acid species (or the nucleic-acid based antibody tag) hybridized to the bead tag. The UMI region can comprise a nucleic acid sequence that provides a counter for the specific occurrence of the target nucleic acid species hybridized to the target-binding region.

In some embodiments, the unique molecular identifier (UMI) sequence is present to correct for any bias and provide accurate quantitative results.

In some embodiments, the target binding region can hybridize with a target of interest. In some embodiments, the target binding region can comprise a nucleic acid sequence that hybridizes specifically to the target nucleic acid with specific gene sequence. In some embodiments, the target binding region can comprise a nucleic acid sequence that can attach (e.g., hybridize) to a specific location of a specific target nucleic acid.

In some embodiments, the target binding regions can comprise a nucleic acid sequence that hybridizes specifically to the nucleic-acid based antibody tag with specific gene sequence. In some embodiments, the target binding region can comprise a nucleic acid sequence that can attach (e.g., hybridize) to a specific location of the nucleic-acid based antibody tag.

In some embodiments, the target binding region can comprise a non-specific target nucleic acid sequence. A non-specific target nucleic acid sequence can refer to a sequence that can bind to multiple target nucleic acids (or the nucleic-acid based antibody tag), independent of the specific sequence of the target nucleic acid (or the nucleic-acid based antibody tag). For example, target binding region can comprise a random multimer sequence, a poly(dA) sequence, a poly(dT) sequence, a poly(dG) sequence, a poly(dC) sequence, or a combination thereof. For example, the target binding region can be an oligo(dT) sequence (e.g., an oligo dT domain) that hybridizes to the poly(A) tail on mRNA molecules. For example, an mRNA molecule can be reverse transcribed using a reverse transcriptase, such as Moloney murine leukemia virus (MMLV) reverse transcriptase, to generate a cDNA molecule with a poly(dC) tail. The barcoded beads can include the target binding region with a poly(dG) tail. Upon base pairing between the poly(dG) tail of the barcode and the poly(dC) tail of the cDNA molecule.

In some embodiments, the target binding region can be an oligo(dT) sequence that hybridizes to a nucleic acid based antibody tag with a poly(A) tail.

In some embodiments, the target binding region can comprise a random multimer sequence. A random multimer sequence can be, for example, a random dimer, trimer, quatramer, pentamer, hexamer, septamer, octamer, nonamer, decamer, or higher multimer sequence of any length (wherein such instances the target binding region may be referred to as a random sequence domain). In some embodiments, the target binding region is a hexamer.

In some embodiments, the target binding region is the same for all bead tags attached to a given bead. In some embodiments, the target binding regions for the plurality of bead tags attached to a given bead can comprise two or more different target binding sequences. A target binding region can be, or be about, 1, 2, 3, 4, 5, 10, 15, 20, 25, 30, 35, 40, 45, 50, or a number or a range between any two of these values, nucleotides in length. A target binding region can be at most about 1, 2, 3, 4, 5, 10, 15, 20, 25, 30, 35, 40, 45, 50 or more nucleotides in length.

In some embodiments, the target binding region can comprise an oligo(dT) (i.e., e.g., an oligo dT domain) which can hybridize with mRNAs comprising polyadenylated ends.

The target binding region can be gene-specific. For example, the target binding region can be configured to hybridize to a specific region of a target (e.g., where the target binding region is a gene specific domain).

In some embodiments, the target binding region can be configured to hybridize to a specific region of a nucleic-acid based antibody tag.

The target binding region can be, or be about, 1, 2, 3, 4, 5, 6, 7, 8, 9, 10, 11, 12, 13, 14, 15, 16, 17, 18, 19, 20, 21, 22, 23, 24, 25, 26 27, 28, 29, 30, or a number or a range between any two of these values, nucleotides in length. The target binding region can be at least, or be at most, 1, 2, 3, 4, 5, 6, 7, 8, 9, 10, 11, 12, 13, 14, 15, 16, 17, 18, 19, 20, 21, 22, 23, 24, 25, 26 27, 28, 29, or 30, nucleotides in length. The target binding region can be about 5-30 nucleotides in length.

The target binding region can interact with a target in a sample. The target can be, or comprise, ribonucleic acids (RNAs), messenger RNAs (mRNAs), microRNAs, small interfering RNAs (siRNAs), RNA degradation products, RNAs each comprising a poly(A) tail, or any combination thereof. In some embodiments, the plurality of targets can include deoxyribonucleic acids (DNAs).

In some embodiments, the target binding region can comprise an oligo(dT) sequence which can interact with poly(A) tails of mRNAs. One or more of the labels of the barcode (e.g., the universal label, the dimension label, the spatial label, the cell label, and the barcode sequences (e.g., molecular label)) can be separated by a spacer from another one or two of the remaining labels of the barcode. The spacer can be, for example, 1, 2, 3, 4, 5, 6, 7, 8, 9, 10, 11, 12, 13, 14, 15, 16, 17, 18, 19, or 20, or more nucleotides. In some embodiments, none of the labels of the barcode is separated by spacer.

Lysis

The methods provided herein include a lysis buffer comprising a lysis reagent. In some embodiments, the lysis reagent is a phenol-based reagent. In some embodiments, the lysis reagent is a guanidinium-based reagent. In some embodiments, the lysis reagent is a high salt-based buffer with or without detergents. In some embodiments, the lysis reagent is a detergent. Detergents suitable for use include, but are not limited to, sodium dodecyl sulfate (SDS), Tween-20, Tween-80, Triton X-100, Nonidet P-40 (NP-40), Brij-35, Brij-58, octyl glucoside, octyl thioglucoside, CHAPS or CHAPSO. In some embodiments, the lysis reagent is QIAzol. In some embodiments, the lysis buffer is M-PER or RIPA buffer.

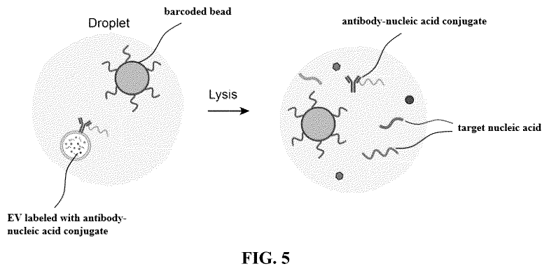

As shown in FIG. 5, the target nucleic acid of the EV is released under lysis buffer. Following lysis of the EV and release of target nucleic acid therefrom, the target nucleic acid can randomly associate with the bead tag.

Association can comprise hybridization of a bead tag's target binding region to a complementary portion of the target nucleic acid (or the nucleic-acid based antibody tag), for instance, oligo(dT) of the target binding region can interact with a poly(A) tail of a target nucleic acid (or the nucleic-acid based antibody tag).

In some embodiments, the target nucleic acid molecules released from the lysed EVs can associate with the plurality of bead tags. When the target binding region comprises oligo(dT), mRNA molecules can hybridize to the target binding region and be reverse transcribed. The oligo(dT) portion of the oligonucleotide can act as a primer for first strand synthesis of the cDNA molecule, e.g., when subject to DNA synthesis reaction conditions to produce first strand cDNA domain comprising capture nucleic acids.

In some embodiments, the target nucleic acid molecule is a cDNA molecule. For example, an mRNA molecule can be reverse transcribed using a reverse transcriptase, such as Moloney murine leukemia virus (MMLV) reverse transcriptase, to generate a cDNA molecule with a poly(dC) tail. A barcoded bead can include a target binding region with a poly(dG) tail the the cDNA molecules can hybridize to the target binding region.

In some embodiments, the target binding region is a random multimer sequence, such as random hexanucleotide primers. Random hexanucleotide primers can bind to target nucleic acids (or the nucleic-acid based antibody tag) at a variety of complementary sites.

In some embodiments, the target binding region is a target-specific oligonucleotide primers which typically selectively prime the target nucleic acids (or the nucleic-acid based antibody tag) of interest.

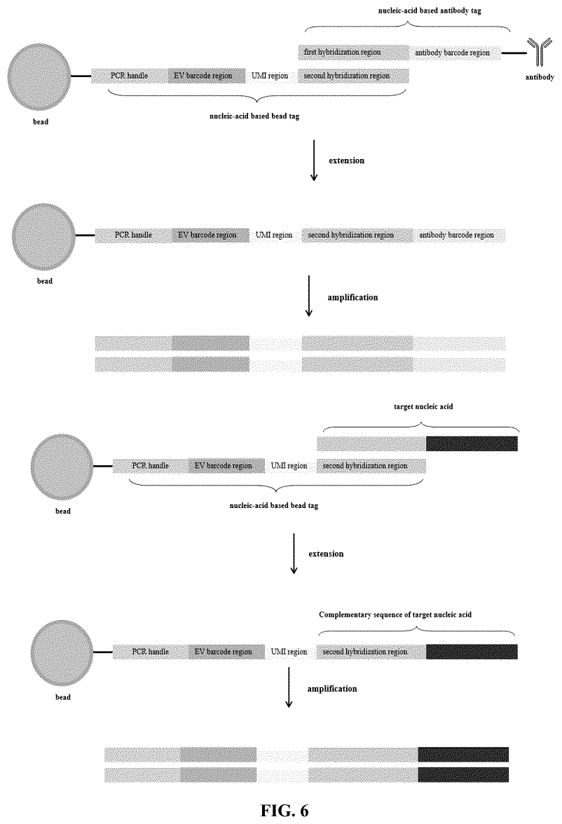

As shown in FIG. 6, followed by the association between the bead tag's target binding region and the target nucleic acid or antibody tag, the complementary paired nucleic acid is extended and amplified. The methods for extension and amplification which may be employed in embodiments of the invention is described in PCT application serial no. PCT/US2020/060692 published as WO/2021/113065, the disclosure of which is herein incorporated by reference.

In one embodiment, the method of multimodal profiling of an EV at single-EV resolution comprises:
a) obtaining a biological sample comprising the EV, wherein the EV contains a surface antigen and a target nucleic acid.
b) incubating the EV with an antibody-nucleic acid conjugate comprising (1) an antibody that binds to the surface antigen, and (2) a nucleic-acid based antibody tag, thereby linking the antibody-nucleic acid conjugate to the EV;
c) purifying the EV to remove excess antibody-nucleic acid conjugate not linking to the EV;
d) incubating the EV with a barcoded bead, wherein the barcoded bead comprise a bead conjugated with a nucleic-acid based bead tag, thereby forming an EV-antibody-nucleic acid conjugate-barcoded bead composition;
e) partitioning the composition in step (d);
f) lysing the partitioned composition in step (e) to release the target nucleic acid and the antibody tag; and
g) sequencing the target nucleic acid, the antibody tag, and the bead tag to profile the EV at single-EV resolution.

In certain embodiments, the EV is derived from neuronal cell and is of low abundance in plasma.

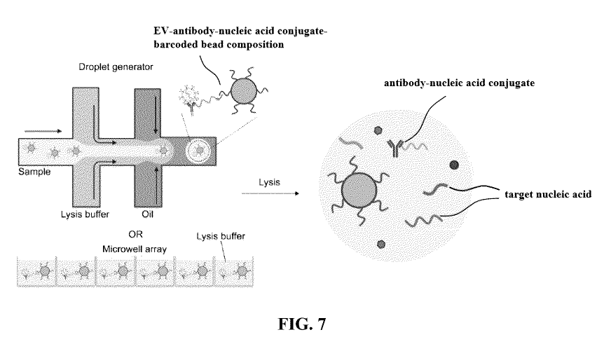

As shown in FIG. 7, the partitioning the composition can be accomplished by droplet generation or separation into microwells.

In certain embodiments, the partitioning step comprises separating the bead by droplet generation. In certain embodiments, the droplet generation comprises a passive droplet generation. In certain embodiments, the droplet generation is accomplished by microfluidics method.

In certain embodiments, the partitioning step comprises separating the composition into microwells. In certain embodiments, separating the composition into the microwells comprises introducing the composition into a support comprising the microwells.

A “microwell” refers to a well having a volume of between 1 n1 to 1000 nL, preferably, between 50 nL to 900 nL, more preferably, between 150 nL to 700 nL, even more preferably, between 250 nL to 600 nL, and most preferably, about 500 nL. The size of a microwell can be selected based on the size of the barcoded beads.

One example of a support comprising microwells is a glass bottom bonded to a silicon grid that creates the micro wells. A support comprising microwells can also be made from poly(dimethylsiloxane) polymer or plastic. Additional materials suitable for preparing a support comprising microwells are known to a skilled artisan and such embodiments are within the purview of the invention.

The methods for introduction of the composition into the microwell which may be employed in embodiments of the invention is described in PCT application serial no. PCT/CN2018/109760 published as WO 2019/068269, the disclosure of which is herein incorporated by reference.

In certain embodiments, the microwells have a size of about 500 nL or less.

To facilitate the introduction of the composition into the microwell, the barcoded bead is a magnetic bead. In certain embodiments, barcoded bead comprises ferrite or magnetite (Fe₃O₄).

In certain embodiments, the ratio of barcoded bead to EV is larger than 1. In certain embodiments, the ratio of barcoded bead to EV is larger than 2. In certain embodiments, the ratio of barcoded bead to EV is larger than 5. In certain embodiments, the ratio of barcoded bead to EV is larger than 10.

In another aspect, the invention further provides device or systems that find use in practicing embodiments of the invention. In some embodiments, the devices or system include a) EXODUS purification module, b) encapsulation module, c) sequencing module and d) data analysis module.

In another aspect, the methods described herein can be used to identify a subject with a disease. In certain embodiments, the subject is a mammal. Non-limiting examples of mammals include human, simian, canine, porcine, bovine, rodent, or feline. In certain embodiments, the disease is cancer. In certain embodiments, the disease is neurodegenerative diseases such as Parkinson's disease (PD) or Alzheimer's disease (AD).

In another aspect, the invention further includes kits that find use in practicing various methods of the invention. The kits may further include one or more additional components finding use in practicing embodiments of the methods. For example, the kits may include one or more of: primers, a polymerase (e.g., a thermostable polymerase, a reverse transcriptase both with hot-start properties, or the like), dsDNAse, exonuclease, dNTPs, a metal cofactor, one or more nuclease inhibitors (e.g., an RNase inhibitor and/or a DNase inhibitor), one or more molecular crowding agents (e.g., polyethylene glycol, or the like), one or more enzyme-stabilizing components (e.g., DTT), a stimulus response polymer, or any other desired kit component(s), such as devices, e.g., as described above, solid supports, containers, cartridges, e.g., tubes, beads, plates, microfluidic chips, etc. Components of the kits may be present in separate containers, or multiple components may be present in a single container.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), portable flash drive, and the like, on which the information has been recorded. Yet another form of these instructions that may be present is a website address which may be used via the internet to access the information at a removed site.

In the Summary of the Invention above and in the Detailed Description of the Invention, and the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or particular claim, that feature can also be used, to the extent possible, in combination with and/or in the context of other particular aspects and embodiments of the invention.

Throughout the present disclosure, the articles “a,” “an,” and “the” are used herein to refer to one or to more than one (i.e., to at least one) of the grammatical object of the article. By way of example, “a compound” means one compound or more than one compound.

As used herein, the term “and/or” indicates that one or more of the stated cases may occur, alone or in combination with at least one of the stated cases, up to with all of the stated cases, and in the invention generally.

The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

Where a range of value is provided, it is understood that each intervening value, to the tenth of the unit of the lower limit unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, the embodiments described herein can be practiced without these specific details. In other instances, methods, procedures and components have not been described in detail so as not to obscure the related relevant function being described. Also, the description is not to be considered as limiting the scope of the implementations described herein. It will be understood that descriptions and characterizations of the embodiments set forth in this disclosure are not to be considered as mutually exclusive, unless otherwise noted.

Unless defined otherwise, all technical and scientific terms used herein have the meaning commonly understood by a person skilled in the art to which this invention belongs. As used herein, the following terms have the meanings ascribed to them unless specified otherwise.

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)—(a second number),” this means a range whose lower limit is the first number and whose upper limit is the second number. For example, 25 to 100 mm means a range whose lower limit is 25 mm, and whose upper limit is 100 mm.

### Example 1

This example illustrates general procedure of the method for multimodal single-EV profiling.

Workflow for Single-EV Profiling

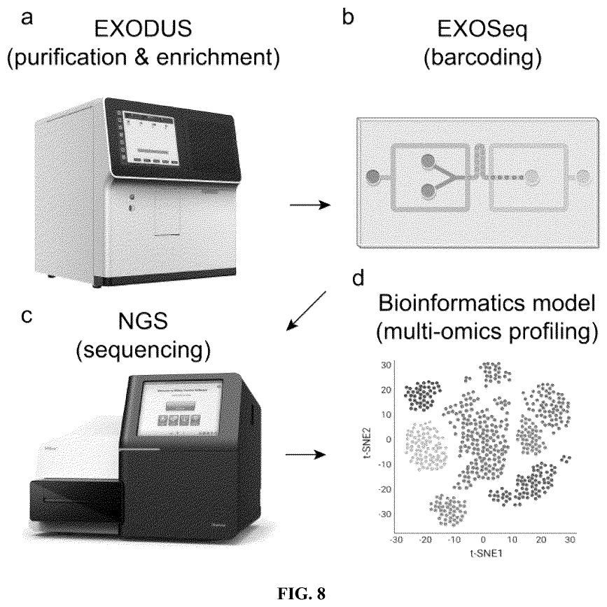

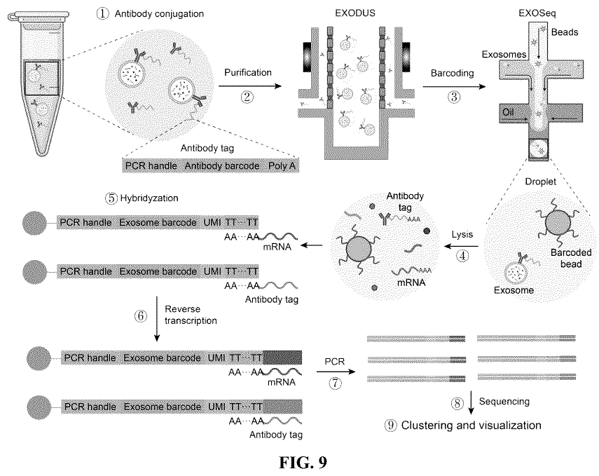

FIG. 8 and FIG. 9 show the workflow for single-EV profiling.

After EV isolation from biofluids via EXODUS, different types of antibodies with unique oligonucleotide tags (antibody barcode) were implemented for conjugation with EV surface proteins, followed by EXODUS purification to remove the unconjugated antibodies and oligonucleotides. The concentration of purified antibody-conjugated EVs was measured by NanoSight (Malvern Panalytical, United Kingdom) and diluted to ˜1000 EVs/μL. Barcoded beads for nucleic acid tagging were diluted with EV lysis buffer into a concentration of ˜1500 particles/μL. To generate droplets, a microfluidic channel (80 um of height and 100 um of width) consisting of a flow-focusing junction was used, where the oil phase converges with the aqueous phase from two sides, “squeezing” the aqueous phase to form droplets. A flow rate of 15 mL/h was used for oil phase, and 4 mL/h was used for both beads and EVs. These parameters were based on previous single-cell studies and further optimized for EVs in our study to achieve high single-particle purity and high conversion rate. Once thousands of emulsion droplets are generated, each with a pair of bead and EV inside, the mRNAs were released as EVs are lysed by the lysis buffer. The mRNAs were then captured by the oligonucleotide tags on beads via the hybridization between poly-A and poly-T tails. After that, all the beads were extracted from the droplets via centrifugation into a uniform aqueous phase for reverse transcription, producing cDNAs labeled with oligonucleotide tags referring to individual EV (EV barcode) and mRNA (UMI, unique molecular identifier). These barcoded cDNAs were amplified via PCR followed by high throughput sequencing for decoding. After identifying the genes, reads were organized by their EV barcodes and individual UMIs were counted for each gene in each EV. The single-EV expression profiles were eventually plotted two dimensionally (tSNE) for visualized analysis to reveal the gene expression relationships between different EV subtypes.

Data Analysis

An open-source Drop-Seq software pipeline was used to analyze the sequencing data. Basically, the cDNA reads were first aligned to a reference genome for gene identification. The genes from the same EVs with same EV barcodes were then grouped. Finally, the expression number of each gene in each EV was counted based on the unique UMIs to create a digital expression matrix. Each EV was two-dimensionally plotted via t-distributed stochastic neighbor embedding (t-SNE) algorithm from the Scikit-learn Library in Python to visualize the high-dimensional data. Similar to gene expression analysis, each protein based on antibody barcodes was identified. The proteins were grouped into the same EVs based on EV barcode, then counted based on unique UMIs as a digital expression matrix for plotting of heap map and t-SNE.

Results and Discussion

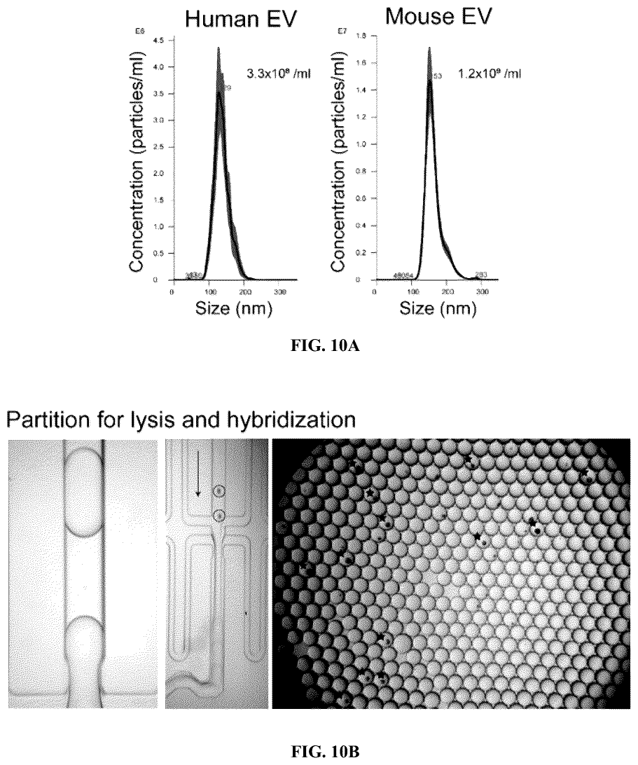

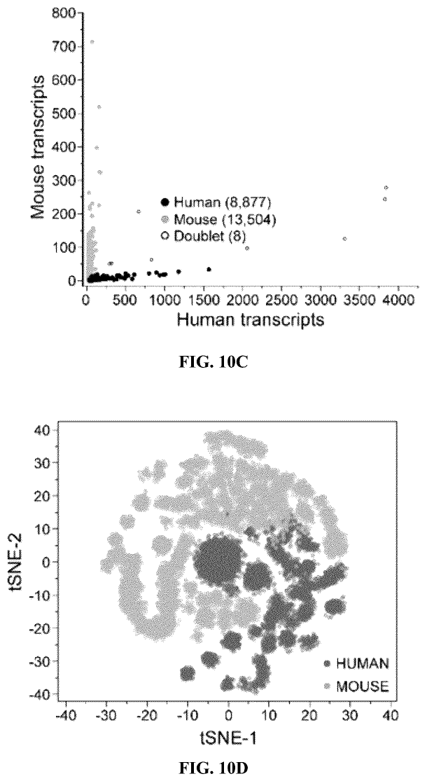

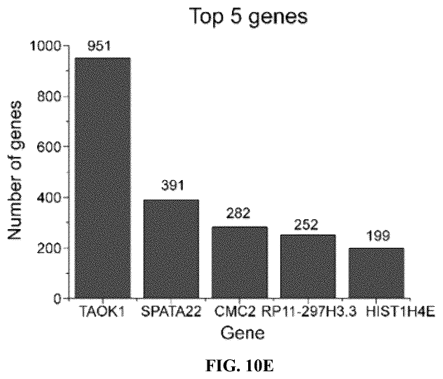

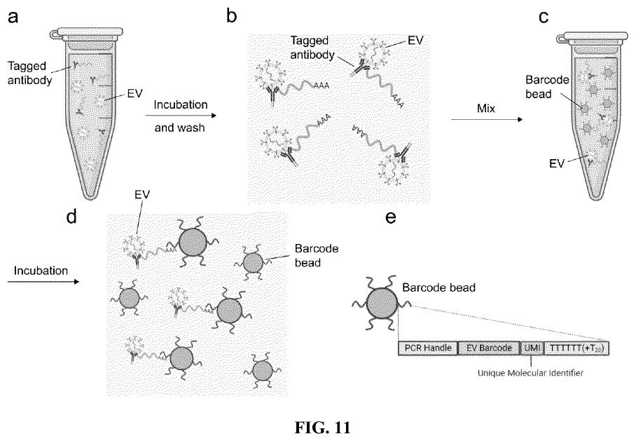

Single-EV RNA sequencing was carried out using a mixture of human HEK and mouse 3T3 cell-derived EVs. After purification via EXODUS, the EVs were measured by NTA (FIG. 10A). Both human and mouse EVs have size ranging from 100-200 nm with concentration above 10⁸/mL. A flow-focusing droplet generator was employed to generate emulsion droplets for encapsulation of single EVs and barcoded beads (FIG. 10B). The single-bead purity was characterized as 95.8% by measuring a small number of droplets under a microscope (FIG. 10B). After reversed transcription and PCR amplification, the cDNA library was purified using SPRI beads. The tagmented library was sequenced using a MiSeq sequencer with the data analyzed via the open-source Drop-Seq software pipeline. The number of individual UMIs was plotted in FIG. 10C. 8,877 human-only transcripts and 13,504 mouse-only transcripts is collected. There were also 8 human-mouse mixed transcripts, indicating a multiplet rate of smaller than 1%, which demonstrates a good performance of the single-EV purity. The digital gene expression (DGE) matrix was employed to generate a two-dimensional t-SNE visualization of the cluster profile, unveiling the transcriptional heterogeneity of single EVs (FIG. 10D). Both human and mouse EVs had multiple subpopulations observed, indicating a high degree of heterogeneity among EVs released by the same cell lines. In addition, the top ranked genes among all EVs were revealed (FIG. 10E).

This example illustrates a method for multimodal profiling of individual EVs. Unlike existing single-EV analysis methods, this method can not only probe exosomal surface proteins, but also profile nucleic acids inside EVs. The droplet-based EV barcoding assay can theoretically analyze unlimited EV surface proteins without mislabeling, which can overcome the limitations of imaging-based methods and proximity barcoding assays. This technique could address the unmet need for multimodal single-EV studies, offering substantial benefits to the EV research community. Its unprecedented resolution and sensitivity for EV interrogation facilitates EV-based biomarker discovery and detection. This method also enables the characterization of EV subpopulations and tissues of origin, as well as improves the understanding of EV biogenesis and biological functions.

### Example 2

This example illustrates that capturing EVs using barcoded beads shows enhanced EV capture efficiency and single-EV purity.

Workflow for Single-EV Profiling

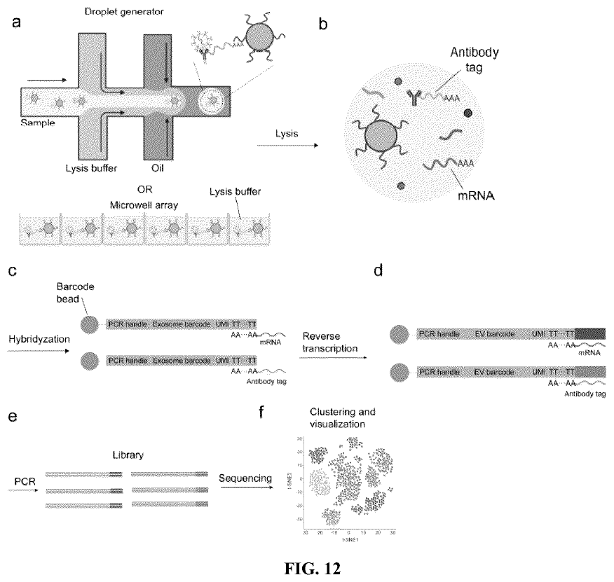

The workflow for single-EV multimodal profiling is shown in FIG. 11 and FIG. 12. The purified EVs were mixed with oligo barcode tagged detection antibodies (FIG. 11a). Each type of antibody had a unique barcode sequence with a poly-dA tail. After incubation and wash to remove the unconjugated antibodies, all the EVs were labeled with barcoded detection antibodies (FIG. 11b). Then the EV sample was mixed with the barcode beads at a concentration ratio of ˜1:10 (FIG. 11c). Each barcode bead had a group of unique oligo barcodes on the surface, which comprising a PCR handle, an EV barcode, a unique molecular identifier (UMI), and a poly-dT tail (FIG. 11e). After incubation for more than 30 min, each EV was captured by one barcode bead via DNA hybridization between poly-dA tail of oligo tags on antibodies and poly-dT tail of oligo barcodes on beads (FIG. 11d). Based on Poisson distribution, some beads capture only one EV (˜10%) and the others had no EV on the surface (˜89%). The chance for one bead to capture two or more EVs was below 1%, ensuring a high EV capture rate and low EV doublet.

The sample then went through a droplet generator or placed into a microwell array, allowing each bead to be encapsulated into a droplet or a microwell with lysis buffer (FIG. 12a). The EVs on bead were then lysed by the lysis buffer (FIG. 12b), allowing the mRNAs and antibody tags with poly-dA to be captured by the oligo barcodes with poly-dT on bead via hybridization (FIG. 12c). After that, a reverse transcription was carried out as shown in FIG. 12d, producing cDNAs labeled with oligonucleotide tags referring to individual EV (EV barcode) and mRNA (UMI, unique molecular identifier). These barcoded cDNAs were amplified via PCR as shown in FIG. 12e followed by high-throughput sequencing for decoding. After identifying the genes, reads were organized by their EV barcodes and individual UMIs were counted for each gene in each EV. The single-EV expression profiles were eventually plotted two-dimensionally (tSNE or UMAP) for visualized analysis to reveal the gene expression relationships between different EV subtypes (FIG. 12f).

Data Analysis

An open-source Drop-Seq software pipeline was used to analyze the sequencing data. Basically, the cDNA reads were first aligned to a reference genome for gene identification. The genes from the same EVs with same EV barcodes were then grouped. Finally, the expression number of each gene in each EV was counted based on the unique UMIs to create a digital expression matrix. Each EV was two-dimensionally plotted via UMAP algorithm from the Seurat Package to visualize the high-dimensional data. Similar to gene expression analysis, each protein based on antibody barcodes was identified. The proteins were grouped into the same EVs based on EV barcode, then counted based on unique UMIs as a digital expression matrix for plotting of heap map and UMAP.

Results and Discussion

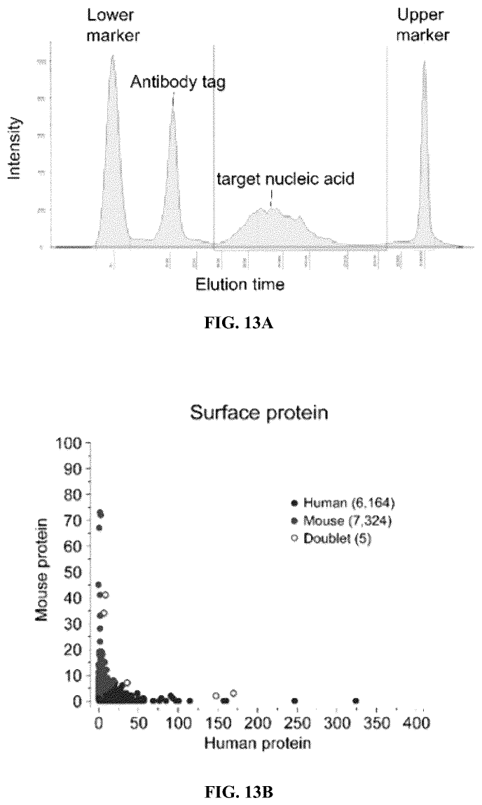

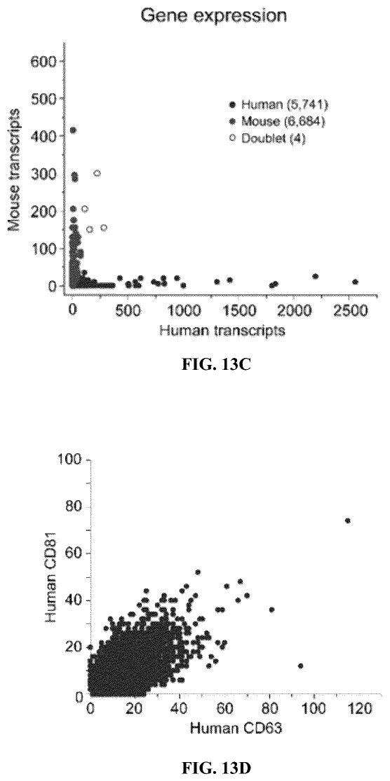

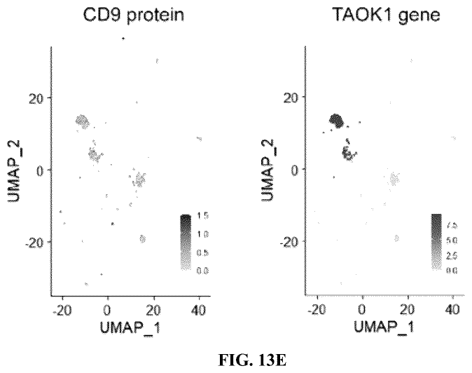

Following the PCR step, the TapeStation analysis yielded a sharp peak at approximately 180 bp, indicating successful amplification of ADTs, and a broad peak ranging from 400 to 2500 bp, indicative of amplified cDNA (FIG. 13A). Subsequently, the sequencing results were aligned to each barcode sequence, enabling the generation of digital gene and protein expression profiles for individual EVs. In FIG. 13B and FIG. 13C, human and mouse protein and transcript numbers were plotted. Both the protein and gene expression profiles showed a low contamination rate (<1%) with doublet occurrences less than 10, indicating high purity of the assay. The number of CD63 and CD81 proteins on human EVs was further plotted (FIG. 13D). A significant majority of human EVs displayed the presence of both CD81 and CD63 markers, exhibiting a distinct distribution pattern when compared to the human-mouse plot. The result further validated the high single-EV and single-bead purity achieved through our assay. Utilizing both protein and gene digital expression matrices, a 2D UMAP clustering visualization was generated for multimodal analysis of individual HEK293 EVs. FIG. 13E shows the expression levels of CD9 protein and TAOK1 gene among different subpopulations. CD9 protein was widely distributed across all EV subpopulations, suggesting its common presence on EVs. In contrast, the TAOK1 genes were found to be selectively carried by specific EV subtypes, indicating distinct expression patterns among the various subpopulations.

The previous description provides exemplary embodiments only, and is not intended to limit the scope, applicability, or configuration of the disclosure. Rather, the previous description of the exemplary embodiments will provide those skilled in the art with an enabling description for implementing one or more exemplary embodiments. It is understood that various changes may be made in the function and arrangement of elements without departing from the spirit and scope of the invention. Several embodiments were described herein, and while various features are ascribed to different embodiments, it should be appreciated that the features described with respect to one embodiment may be incorporated within other embodiments as well. By the same token, however, no single feature or features of any described embodiment should be considered essential to every embodiment of the invention, as other embodiments of the invention may omit such features.

Specific details are given in the previous description to provide a thorough understanding of the embodiments. However, it will be understood by one of ordinary skill in the art that the embodiments may be practiced without these specific details. For example, circuits, systems, networks, processes, and other elements in the invention may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process may be terminated when its operations are completed, but could have also included additional steps or operations not discussed or included in a figure.

Furthermore, not all operations in any particularly described process may occur in all embodiments. A process may correspond to a method, a function, a procedure, a subroutine, a subprogram, etc. When a process corresponds to a function, its termination corresponds to a return of the function to the calling function or the main function.

Furthermore, embodiments may be implemented, at least in part, either manually or automatically. Manual or automatic implementations may be executed, or at least assisted, through the use of machines, hardware, software, firmware, middleware, microcode, hardware description languages, or any combination thereof. When implemented in software, firmware, middleware or microcode, the program code or code segments to perform the necessary tasks may be stored in a machine readable medium. A processor(s) may perform the necessary tasks.

While detailed descriptions of one or more embodiments have been give above, various alternatives, modifications, and equivalents will be apparent to those skilled in the art without varying from the spirit of the invention. Moreover, except where clearly inappropriate or otherwise expressly noted, it should be assumed that the features, devices, and/or components of different embodiments may be substituted and/or combined. Thus, the above description should not be taken as limiting the scope of the invention. Lastly, one or more elements of one or more embodiments may be combined with one or more elements of one or more other embodiments without departing from the scope of the invention.

### REFERENCES

1 Kalluri, R. & LeBleu, V. S. The biology, function, and biomedical applications of exosomes. Science 367 (2020).
2 Zhang, Y., Liu, Y., Liu, H. & Tang, W. H. Exosomes: biogenesis, biologic function and clinical potential. Cell Biosci 9, 19 (2019).
3 Chen, C. et al. Profiling of Exosomal Biomarkers for Accurate Cancer Identification: Combining DNA-PAINT with Machine-Learning-Based Classification. Small 15, e1901014 (2019).
4 Jia, Y. et al. Exosome: emerging biomarker in breast cancer. Oncotarget 8, 41717-41733 (2017).
5 Xu, K. et al. Extracellular vesicles as potential biomarkers and therapeutic approaches in autoimmune diseases. J Transl Med 18, 432 (2020).
6 Badhwar, A. & Haqqani, A. S. Biomarker potential of brain-secreted extracellular vesicles in blood in Alzheimer's disease. Alzheimers Dement (Amst) 12, e12001 (2020).
7 Mastoridis, S. et al. Multiparametric Analysis of Circulating Exosomes and Other Small Extracellular Vesicles by Advanced Imaging Flow Cytometry. Front Immunol 9, 1583 (2018).
8 Daaboul, G. G. et al. Digital Detection of Exosomes by Interferometric Imaging. Sci Rep 6, 37246 (2016).
9 Wu, D. et al. Profiling surface proteins on individual exosomes using a proximity barcoding assay. Nat Commun 10, 3854 (2019).
10 Liang, K. et al. Nanoplasmonic Quantification of Tumor-derived Extracellular Vesicles in Plasma Microsamples for Diagnosis and Treatment Monitoring. Nat Biomed Eng 1 (2017).
11 Stuart, T. & Satija, R. Integrative single-cell analysis. Nat Rev Genet 20, 257-272 (2019).
12 Chen, G., Ning, B. & Shi, T. Single-Cell RNA-Seq Technologies and Related Computational Data Analysis. Front Genet 10, 317 (2019).
13 Chiang, C. Y. & Chen, C. Toward characterizing extracellular vesicles at a single-particle level. J Biomed Sci 26, 9 (2019).
14 Rajagopal, C. & Harikumar, K. B. The Origin and Functions of Exosomes in Cancer. Front Oncol 8, 66 (2018).
15 Maia, J., Caja, S., Strano Moraes, M. C., Couto, N. & Costa-Silva, B. Exosome-Based Cell-Cell Communication in the Tumor Microenvironment. Front Cell Dev Biol 6, 18 (2018).
16 Chang, C. H. et al. Evaluation of digital real-time PCR assay as a molecular diagnostic tool for single-cell analysis. Sci Rep 8, 3432 (2018).
17 Hague, A., Engel, J., Teichmann, S. A. & Lonnberg, T. A practical guide to single-cell RNA-sequencing for biomedical research and clinical applications. Genome Med 9, 75 (2017).
18 Simonsen, J. B. What Are We Looking At? Extracellular Vesicles, Lipoproteins, or Both? Circ Res 121, 920-922 (2017).
19 Lane, R. E., Korbie, D., Hill, M. M. & Trau, M. Extracellular vesicles as circulating cancer biomarkers: opportunities and challenges. Clin Transl Med 7, 14 (2018).
20 Chen, Y. et al. Exosome detection via the ultrafast-isolation system: EXODUS. Nat Methods 18, 212-218 (2021).
21 Macosko, E. Z. et al. Highly Parallel Genome-wide Expression Profiling of Individual Cells Using Nanoliter Droplets. Cell 161, 1202-1214 (2015).
22 Bageritz, J. & Raddi, G. Single-Cell RNA Sequencing with Drop-Seq. Methods Mol Biol 1979, 73-85 (2019).
23 Klein, A. M. & Macosko, E. InDrops and Drop-seq technologies for single-cell sequencing. Lab Chip 17, 2540-2541 (2017).
24 Klein, A. M. et al. Droplet barcoding for single-cell transcriptomics applied to embryonic stem cells. Cell 161, 1187-1201 (2015).
25 Behjati Ardakani, F. et al. Integrative analysis of single-cell expression data reveals distinct regulatory states in bidirectional promoters. Epigenetics Chromatin 11, 66 (2018).
26 Yamawaki, T. M. et al. Systematic comparison of high-throughput single-cell RNA-seq methods for immune cell profiling. BMC Genomics 22, 66 (2021).
27 Method of the Year 2020: spatially resolved transcriptomics. Nat Methods 18, 1 (2021).

What is claimed is:

1. A method of multimodal profiling of an extracellular vesicle (EV) at single-EV resolution, the method comprising:
a) obtaining a biological sample comprising the EV, wherein the EV contains a surface antigen and a target nucleic acid;
b) incubating the EV with an antibody-nucleic acid conjugate comprising (1) an antibody that binds to the surface antigen, and (2) a nucleic-acid based antibody tag, thereby linking the antibody-nucleic acid conjugate to the EV;
c) purifying the EV to remove excess antibody-nucleic acid conjugate not linking to the EV;
d) mixing the labeled EV, a barcoded bead, and an EV lysis buffer, wherein the barcoded bead comprise a bead conjugated with a nucleic-acid based bead tag;
e) lysing the EV to release the target nucleic acid and the antibody tag; and
f) sequencing the target nucleic acid, the antibody tag, and the bead tag to profile the EV at single-EV resolution.

2. The method of claim 1, wherein the step (d) further comprising encapsulating the labeled EV, the barcoded bead, and the EV lysis buffer into a droplet.

3. The method of claim 2, wherein the target nucleic acid and the antibody tag is released to the droplet.

4. The method of claim 1, wherein in the step (d) the EV is further linked to the barcoded bead.

5. The method of claim 4, wherein the method further comprising partitioning the EV-bead conjugate into a droplet or a microwell with EV lysis buffer after the step (d).

6. The method of claim 5, wherein the target nucleic acid and the antibody tag is released to the droplet or the microwell.

7. The method of claim 1, wherein the target nucleic acid is mRNA, microRNA, lncRNA, tRNA, snRNA, YRNA, or vault RNA.

8. The method of claim 1, wherein the target nucleic acid is mRNA.

9. The method of claim 1, wherein the surface antigen is selected from the group consisting of CD56, CD171, CD9, CD63, CD81, GPC1, FN, PSMA, CD30, FoxP3, CCR8, SiglecF, Ly6G, CCL3, ga13, t-Tau, p-Tau, Aβ40, and Aβ42.

10. The method of claim 1, the antibody tag comprises an antibody barcode region, and an antibody hybridization region.

11. The method of claim 1, wherein the antibody hybridization region is a poly dA tail.

12. The method of claim 1, wherein the purifying step involves an ultrafiltration-based method.

13. The method of claim 1, wherein the ultrafiltration-based method is tangential flow filtration.

14. The method of claim 1, wherein the ultrafiltration-based method is EXODUS (EV detection via the ultrafast-isolation system).

15. The method of claim 2, wherein the labeled EV, the barcoded bead, and the EV lysis is encapsulated using a microfluidics process.

16. The method of claim 15, wherein the ratio of the barcoded bead to labeled EV is about 1.5

17. The method of claim 15, wherein the labeled EV is at a concentration of about 1000 EV/μL and the barcoded bead is at a concentration of 1500 beads/μL in the lysis buffer.

18. The method of claim 15, wherein the microfluidic process uses an oil phase having a flow rate of 15 mL/h.

19. The method of claim 1, wherein the nucleic-acid based bead tag comprises a PCR handle, an EV barcode region, a unique molecular identifier (UMI) region and a target binding region.

20. The method of claim 1, wherein the target binding region is a poly dT tail, or a random sequence, or a combination of both.

21. The method of claim 1, wherein in the step (e) the target nucleic acid and the antibody tag are captured by the barcode bead through the target binding region.

22. The method of claim 1, wherein in the step (f) the target nucleic acid and antibody tag are amplified via polymerase chain reaction before sequencing (PCR).

23. The method of claim 22, wherein the PCR products comprise the sequence information from nucleic acid on barcoded bead and the sequence information from target nucleic or antibody tag.

24. The method of claim 1, wherein the barcoded bead is a magnetic bead, optionally wherein the barcoded bead comprises ferrite or magnetite (Fe₃O₄)

25. The method of claim 4, wherein the ratio of the barcoded bead to the EV is larger than 1, 2, 5 or 10.

26. The method of claim 5, wherein the EV is partitioned into the droplet using a microfluidics process.
